(19) United States
(12) Patent Application Publication
(10) Pub. No.: US 2026/0308775 A1
(43) Pub. Date: Oct. 8, 2026

(54) CO2 AND H2O CO-ELECTROLYSER SYSTEM

(71) Applicant: ECOLE POLYTECHNIQUE FEDERALE DE LAUSANNE (EPFL), Lausanne (CH)

(72) Inventors: Rafael AMORIM LEANDRO DE CASTRO AMOEDO, ZÜRICH (CH); Suhas NUGGEHALLI SAMPATHKUMAR, SION (CH); François MARÉCHAL, SION (CH); Jan VAN HERLE, LONAY (CH); Tommaso FRONTALI, Rome (IT)

(21) Appl. No.: 19/484,785

(22) PCT Filed: May 14, 2024
(86) PCT No.: PCT/EP2024/063213

§ 371 Date: Nov. 14, 2025

(30) Foreign Application Priority Data
May 17, 2023 (EP) 23173892.3

(51) Int. Cl. C25B 1/23; B01D 53/047; C01B 32/60; C01F 5/24; C07C 1/04; C10L 3/08; C25B 1/04; C25B 9/23; C25B 15/08

(52) U.S. Cl. CPC C25B 1/23; B01D 53/047; C01B 32/60; C01F 5/24; C07C 1/041; C10L 3/08; C25B 1/04; C25B 9/23; C25B 15/081; C25B 15/083; C25B 15/087; B01D 2256/22; B01D 2257/104; C10L 2290/38; C10L 2290/42

(57) ABSTRACT

A water and carbon dioxide co-electrolyser system (1) comprising an anion exchange membrane (AEM) electrolyser (2) having at least one AEM electrolyser cell (2c) comprising a cathode (8), an anode (12) and an AEM membrane (16) separating the cathode from the anode, and an anolyte circuit (18) in which an anolyte flows connected to the anode (12) via an anolyte inlet (14i) and anolyte outlet (140) of the anode (12). The CO₂ and H₂G co-electrolyser system further comprises a mineralization system (3) including a mineralization unit (27) connected to the anolyte circuit (18) and containing a mineralization metal configured to react with the carbonate and bicarbonate ions circulating in the anolyte circuit (18) to form a metal carbonate.

The present invention relates to a system for co-electrolysis of CO₂ and H₂O, in particular for the production of fuels.

Promoting the use and conversion of carbon dioxide (CO₂) is a major goal in the upcoming decades.

CO₂ electrolysis has recently grown in interest for its potential to produce low-carbon fuels using green energy. Carbon dioxide can be reduced into a vast number of products, such as carbon monoxide (CO), syngas (CO+H₂), methane (CH₄), formic acid (HCOOH), ethylene (C₂H₄), methanol (CH₃OH), ethanol (C₂H₆O) or n-propanol (C₃H₈O), depending on the number of electrons required for the reaction and on the catalyst used. CO₂ electro-reduction to CO (or syngas) is one of the economically competitive processes. The redox reaction of CO₂ to CO requires a minimum amount of energy to occur, represented thermodynamically by the reaction enthalpy of formation AHr. This energy is built up of two contributions: electrical energy, represented by the Gibbs free energy change AGr, and heat, represented by the entropy change TASr. The extent to which each factor influences the process depends mainly on the operating temperature and partial pressure whereby high-temperature electrolyzers need a lower electricity input and a higher heat supply with respect to low-temperature electrolyzers.

High-temperature (i.e. >600° C.) electrolysis technology using a Solid-oxide electrolysis cell (SOEC) is known, but has certain drawbacks associated to the use of critical resource materials for the membrane electrode assembly, the durability and stability of the membrane subjected inter alia to thermal stresses especially in transitional operation (e.g. start-up and shut down) due to the high operating temperature, and the overall efficiency of the CO₂ and H₂O conversion process. There is a continuous desire to improve high reaction rates and energy efficiency for the electrochemical reduction of CO₂ to value-added products such as fuels and chemical, to be cost-competitive and achieve industrial deployment.

Low-temperature (<=200° C.) electrolyzers would offer greater operation flexibility and overcome some of the drawbacks of SOEC electrolysers related to low current densities, fragility under pressurisation and high temperature operation.

Typical CO₂ electrolytic cells comprise two electrodes, cathode and anode, where the CO₂ Reduction Reaction (CO₂RR) and Oxygen Evolution Reaction (OER) occur, respectively, promoted by catalysts. Cathode and anode are separated by an ion-exchange membrane, which allows ion transfer and avoids product crossover. Product selectivity mainly depends on the catalyst type and the environment's pH. Ag, Cu, Au, Sn, and bimetallic Cu/Sn catalysts are often used for CO₂ electro-reduction. In particular, Ag and Au are highly selective toward carbon monoxide production in alkaline conditions. Depending on the membrane's ionic permeability, three types of membranes that operate at low temperatures and could be used to electrochemically convert CO₂ into fuels and chemicals at low-temperatures, have been classified: Anion-exchange membrane (AEM), Cation-exchange membrane (CEM) and Bipolar membrane (BPM). AEM and CEM can conduct anions and cations/protons, respectively, while BPM is a combination of the previous two and hence can conduct both anions and cations.

AEM cells have so far demonstrated superior performances in terms of product selectivity and stability with respect to the other membranes. However, one of the important limitations of AEM membranes for CO₂ conversion derives from the cross-over of charged species such as CO₃²⁻ and HCO₃⁻, which drastically reduces CO₂ conversion and thus system efficiency.

In a CO₂—H₂O to syngas co-electrolysis cell, CO₂RR (Eq.1.1), together with the competing Hydrogen Evolution Reaction (HER) (Eq.1.2), occur at the cathode, bringing to the formation of hydroxide ions (OH—), while OER occur at the anode (Eq 1.3 and/or Eq 1.4).

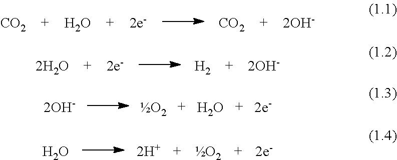

In a CO₂ rich environment, the formed OH— react with the CO₂ in excess (Eq.1.5, 1.6) producing bicarbonate (HCO⁻₃) and carbonate (CO²⁻₃) ions. These negatively charged ions can cross the membrane and afterward release CO₂ at the anode (Eq. 1.7, 1.8), reducing the total CO₂ conversion. This process is also known as the “CO₂ pumping effect” since for each CO₂ being reduced to CO₂ one or two CO₂ molecules are released at the anode, depending on the major charge carrier through the membrane being carbonate or bicarbonate ions, respectively.

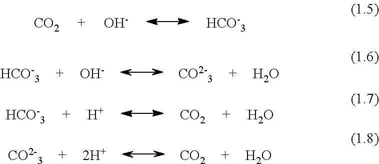

Another problem is that cations can be transported from the anode to the cathode due to a concentration gradient and can react with carbonate/bicarbonate ions formed from the CO₂RR, causing salt precipitation and damaging the cell.

CO₂ and products from CO₂ conversion may be used for producing fuels and chemical products. One of the known processes is a methanation process. CO₂ methanation, also known as Sabatier reaction (Eq.1.9), is an highly exothermic reaction (ΔHr=−165.1 kJ/mol) that can also be seen as a combination of two reactions: the exothermic (ΔHr=−206.3 kJ/mol) CO methanation (Eq.1.10) and the endothermic (ΔHr=41.2 kJ/mol) Reverse Water Gas Shift (RWGS) reaction (Eq.1.11) [51, 52]:

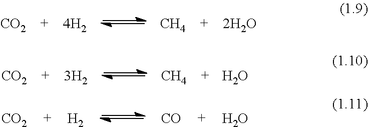

Thermodynamically, the reaction is favored in a temperature range between 20° and 500° C. and at high pressure (1-100 bar). The maximum CH₄ production is obtained with a H₂/(CO+CO₂) ratio close to the stoichiometric one. To hinder undesired competing reactions, it is essential to remove the heat produced and maintain the temperature inside a specific range in the methanation reactor. This removed heat energy can however be used and integrated into the system to improve efficiency.

In view of the foregoing, it is an object of this invention to provide a water and carbon dioxide co-electrolyser system that has a high carbon dioxide conversion rate and yield, yet is energy efficient and economical.

It is advantageous to provide a water and carbon dioxide co-electrolyser system that is economical to produce and to operate.

It is advantageous to provide a water and carbon dioxide co-electrolyser system that is reliable and durable.

It is advantageous to provide a water and carbon dioxide co-electrolyser system that is usable in various configurations and in particular that can be connected to renewable energy sources.

Another aim of the invention, which concerns one of the main applications of interest, is to provide a synthetic natural gas (SNG) production system that has a high carbon dioxide conversion rate and yield, and is globally efficient and economical.

Objects of this invention have been achieved by providing a water and carbon dioxide co-electrolyser system according to claim 1, and a SNG production system according to claim 13. Dependent claims set forth various advantageous features of embodiments of the invention.

Disclosed herein is a water and carbon dioxide co-electrolyser system comprising
an anion exchange membrane (AEM) electrolyser having at least one AEM electrolyser cell comprising a cathode, an anode and an AEM membrane separating the cathode from the anode, and
an anolyte circuit in which an anolyte flows connected to the anode via an anolyte inlet and anolyte outlet of the anode.

The CO₂ and H₂O co-electrolyser system further comprises a mineralization system including a mineralization unit connected to the anolyte circuit and containing a mineralization metal configured to react with the carbonate and bicarbonate ions circulating in the anolyte circuit to form a metal carbonate.

The invention thus turns the disadvantage of the generation of bicarbonate (HCO⁻₃) and carbonate (CO₂⁻₃) ions for and AEM electrolyser into an asset: the crossover species are used to produce carbonates, i.e. stable formations that harvest CO₂ in their matrix, thus promoting CO₂ removal and storage in a stable form, while at the same time producing syngas (CO+H₂) which can be valorized in a reactor to form methane.

In an advantageous embodiment, the anolyte circuit forms a closed circuit for recycling the anolyte solution.

In an advantageous embodiment, the anolyte circuit comprises an anolyte recycling tank having a water inlet and an alkaline solution inlet for replenishing the anolyte.

In an advantageous embodiment, the mineralization system comprises a metal carbonate deposit outlet at a bottom of a vessel of the mineralization unit for removal of metal carbonate deposits from the mineralization unit.

In an advantageous embodiment, the mineralization system comprises a mineralization metal inlet configured for injection of a solution containing metal ions into the anolyte circuit for reaction with the carbonate and bicarbonate ions to produce a metal carbonate that is precipitated out of the anolyte solution.

In a variant, the mineralization metal is provided in the form of a solid element such as a rod, bar, plate, or mesh.

The mineralization metal may be selected from a group of metals consisting of Li, Na, K, Mg, Ca, Mn, Fe, Co, Ni, Cu, Zn.

In an embodiment, the mineralization metal is selected from a group of metals consisting of Mg, Ca, Mn, Fe, Co, Ni, Cu, Zn.

In an advantageous embodiment, the mineralization metal is selected from a group of metals consisting of Mg, Ca.

In an advantageous embodiment, the AEM membrane comprises one or more layers of polystyrene methyl methylimidazolium chloride (PSMIM) or polystyrene tetramethyl methylimidazolium chloride (PSTMIM) or functionalized poly (aryl piperidinium) polymer, sandwiched between one or more catalyst layers.

In an advantageous embodiment, the AEM electrolyser anode comprises an anode products outlet connected to a gas separator, for instance comprising a pressure swing absorption (PSA) unit for separating carbon dioxide and oxygen output from the anode.

In an advantageous embodiment, the cathode is connected to a carbon dioxide source and a water source that are connected to a humidifier for injecting humidified carbon dioxide into the cathode side of the AEM electrolyser.

Also disclosed herein, is a synthetic natural gas (SNG) production system comprising a carbon dioxide and water co-electrolyser system according to any of the above embodiments and further comprising a methanation system including a methanation reactor connected to the outlet of the cathode and an outlet of the gas separator outputting carbon dioxide.

In an advantageous embodiment, the SNG production system further comprises a hydrogen source including a water electrolyser connected preferably to a renewable energy electrical source

Further objects and advantageous features of the invention will be apparent from the claims, from the detailed description, and annexed drawings, in which:

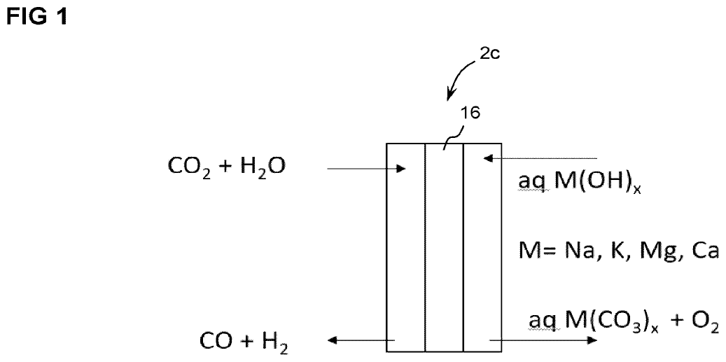

FIG. 1 is a simplified schematic illustration of an anion exchange membrane (AEM) electrolyser cell showing the main chemical products output by the cell;

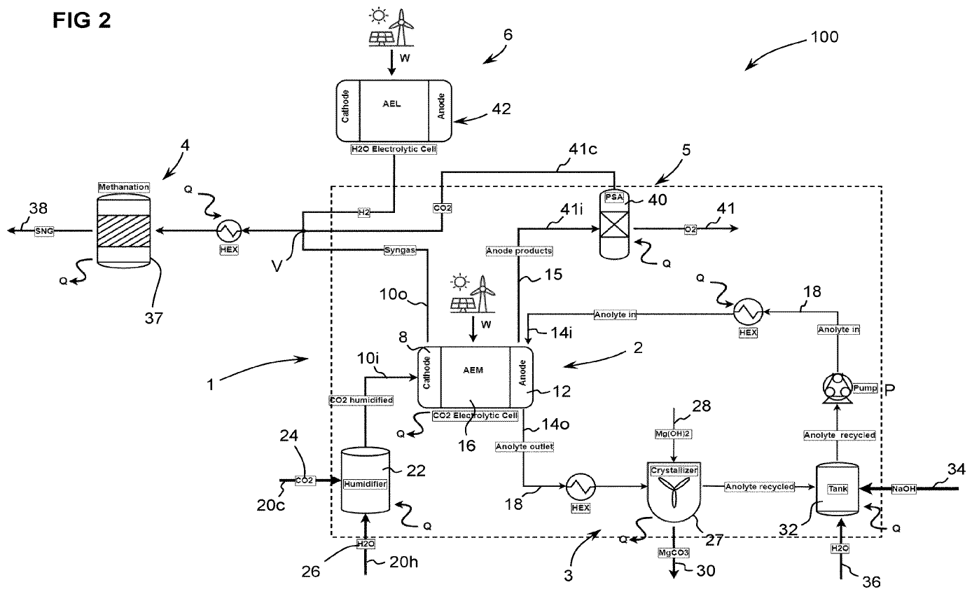

FIG. 2 is a schematic diagram of a synthetic natural gas (SNG) production system with a CO₂ and H₂O co-electrolyser system and a mineralization system according to an embodiment of the invention;

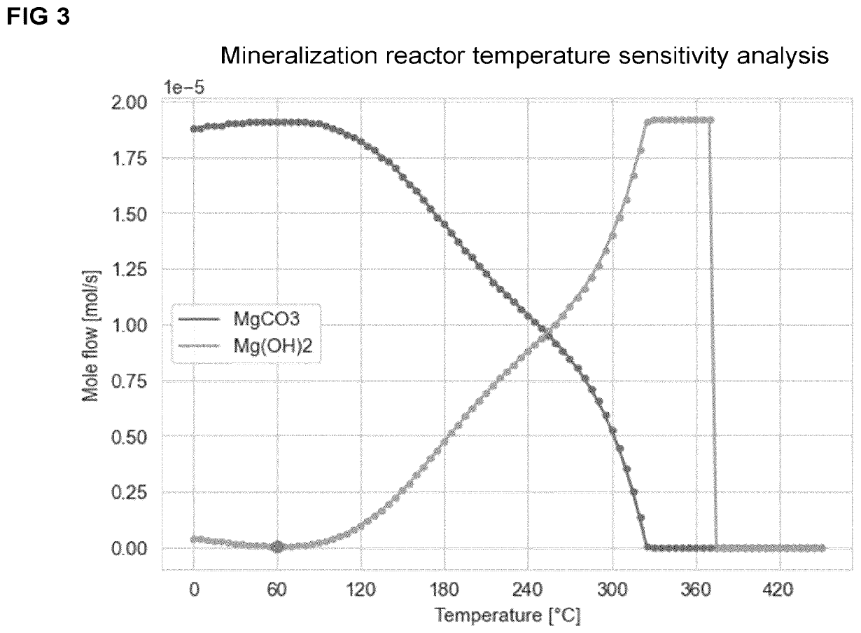

FIG. 3 is plot of flow versus temperature illustrating a mineralization reactor temperature sensitivity analysis of a mineralization system according to an embodiment of the invention;

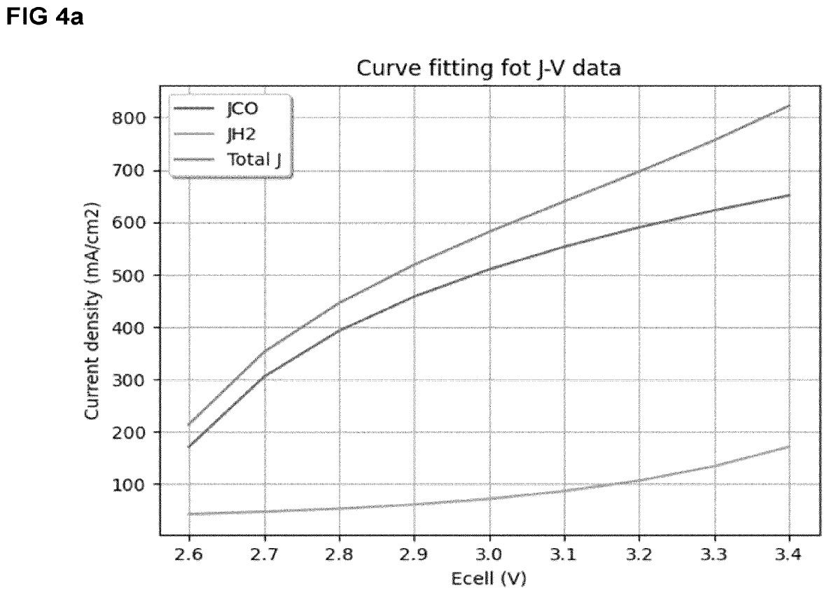

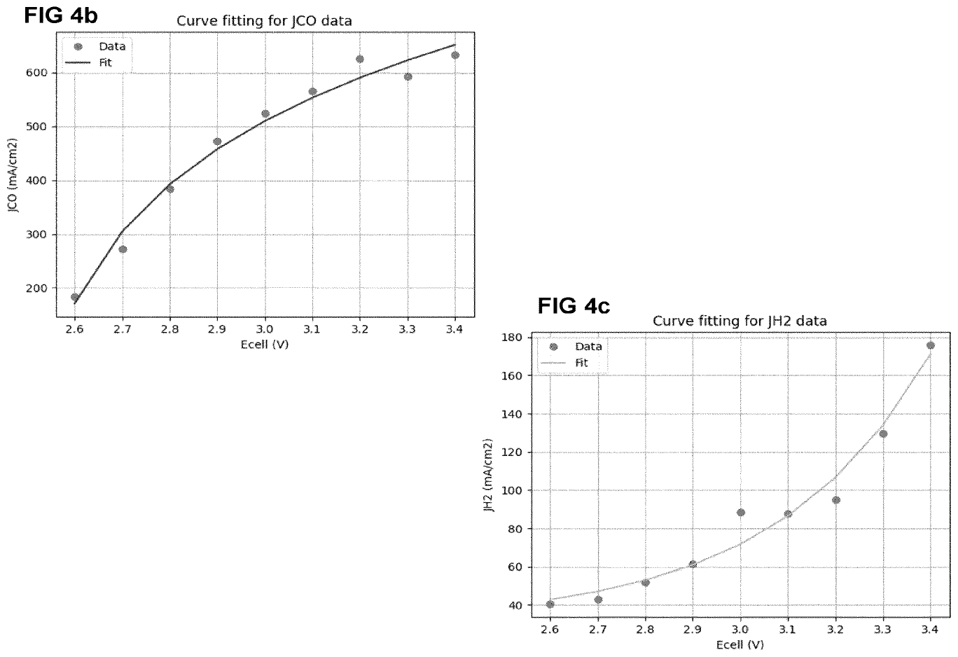

FIG. 4a is a plot of current density versus voltage of a co-electrolyser cell of an anion exchange membrane according to an embodiment of the invention;

FIG. 4b is a plot similar to FIG. 4a illustrating data obtained for carbon monoxide conversion;

FIG. 4c a plot similar to FIG. 4a illustrating data obtained for hydrogen conversion;

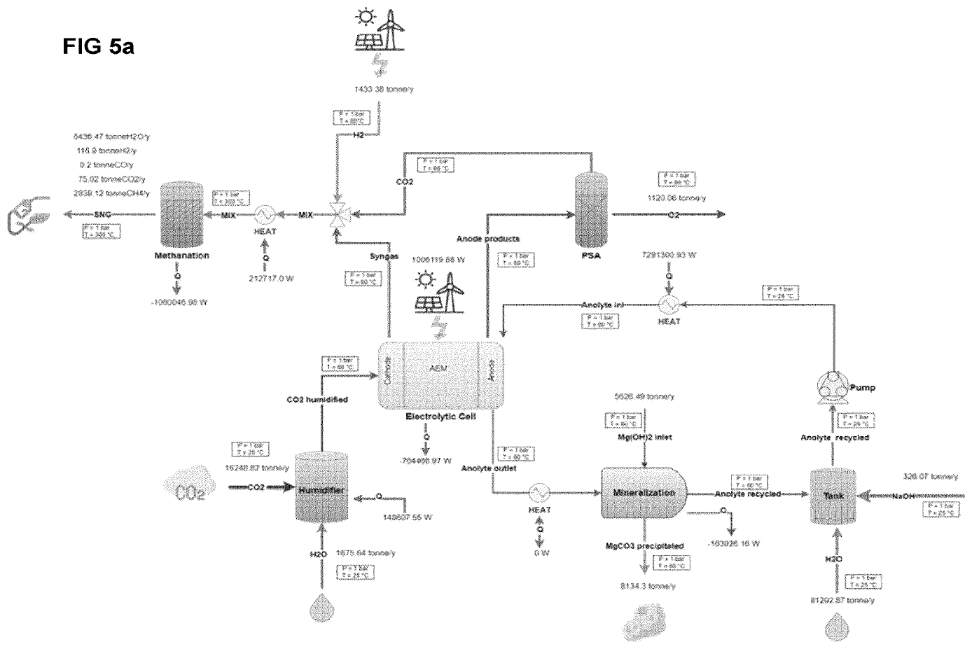

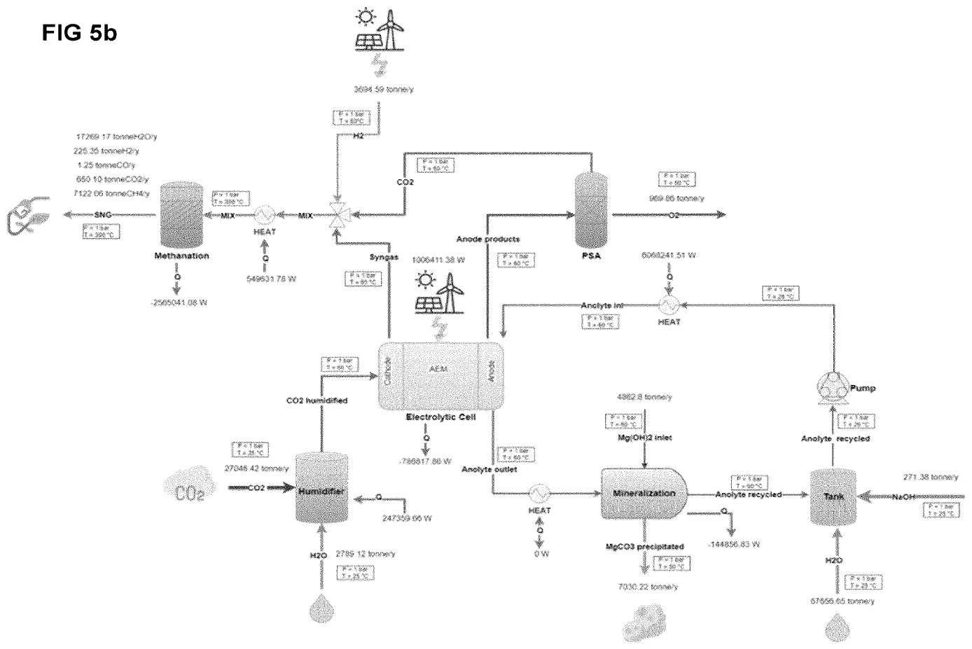

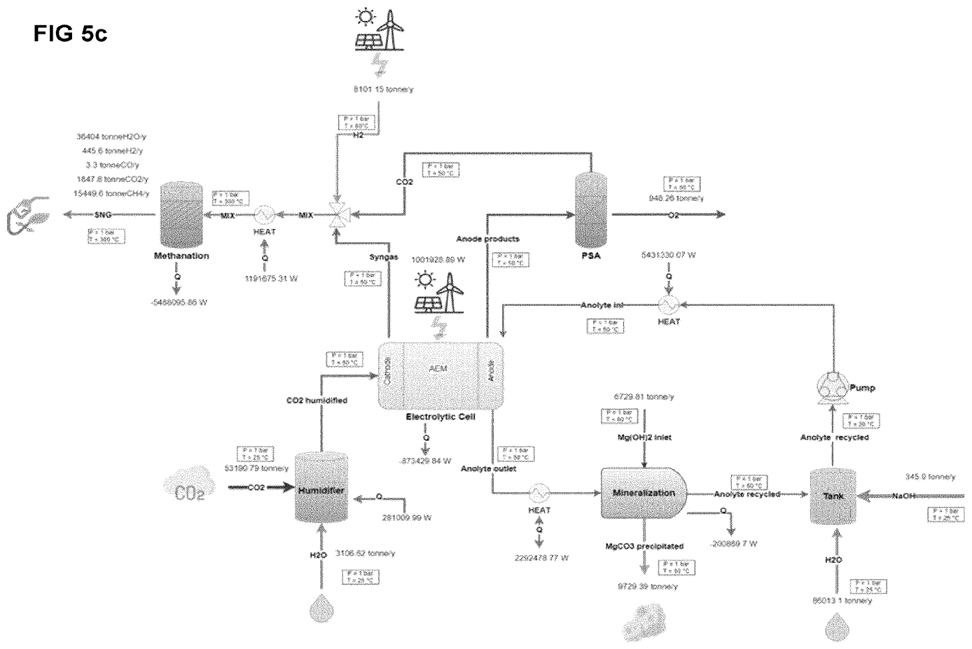

FIGS. 5a to 5c illustrate the system of FIG. 2 with examples of different gas and energy inputs, and resulting outputs, representing different use cases and configurations of the SNG production system.

One global aspect of the invention is the provision of a Power to Synthetic Natural Gas (PtSNG) system comprising an anion exchange membrane (AEM) electrolyser configured for CO₂/H₂O co-electrolysis, and a mineralization system to produce SNG and a metal carbonate such as magnesium carbonate, simultaneously.

Referring to the figures, in particular FIG. 2, a SNG production system 100 comprises a CO₂/H₂O co-electrolyser system 1 comprising an electrolyser 2, a carbon dioxide source 24, a water source 26, an anolyte circuit 18, an anode products separation system 5, and a mineralization system 3. The SNG production system further comprises a methanation system 4. The SNG production system may further comprise a hydrogen source 6 that may in particular comprise a water electrolyser system coupled to a renewable source of electrical energy.

According to an aspect of the invention, the electrolyser 2 comprises an anion exchange membrane (AEM) electrolyser cell 2c having a cathode 8, an anode 12, and an AEM membrane 16 between the cathode and anode. AEM membranes are per se known and commercially available, materials of various such membranes including polystyrene methyl methylimidazolium chloride (PSMIM), polystyrene tetramethyl methylimidazolium chloride (PSTMIM) and functionalized poly (aryl piperidinium) polymer, sandwiched between one or more catalyst layers.

The cathode 8 has an inlet 10i connected to the carbon dioxide source 24 and water source 26 whereby the carbon dioxide and water sources may be fed into a humidifier 22 that produces a humidified CO₂ gas, in other words a mixture of H₂O and CO₂ gas fed into the cathode of the AEM electrolyser cell. The cathode comprises an outlet 10o that outputs the cathode gas products comprising in particular synthetic natural gas consisting mainly of carbon monoxide (CO) and hydrogen (H₂) gas.

The anode 12 of the electrolyser 2 comprises an anolyte inlet 14i connected to the anolyte circuit 18, and an anolyte outlet 14o connected to the anolyte circuit 18. The anode 12 or anolyte circuit 18 further comprises an anode products outlet 15 that is connected to an anode products separator system 5 which in particular may comprise a gas separator for instance in the form of a pressure swing absorption (PSA) reactor 40 having an anode products inlet 41i connected to the anode products system 15 of the anode 12. The anode products comprise principally oxygen and residual carbon dioxide that is processed by the PSA unit 40 to output oxygen via an oxygen outlet 41o and CO₂ via a CO₂ outlet 41c that is fed to the methanation system 4.

The mineralization system 3 is configured to remove the carbonate and bicarbonate ions formed at the cathode and crossing the membrane to the anode, by mineralization with a metal in a mineralization unit 27 of the mineralization system.

The anolyte circuit 18 comprises a closed-loop circuit where the anolyte containing the carbonates and bicarbonates flows out through the outlet 14o into the mineralization unit 27, the carbonates reacting with a metal (e.g. Magnesium (Mg)) to produce a metal carbonate (e.g. MgCO₃), and the anolyte is recycled back to the anode of the electrolyser 2. The metal carbonate may be extracted for instance via a metal carbonate outlet 30 of the mineralization system 3, for instance in a batch wise manner. The metal for the mineralization of the carbonate and bicarbonate ions may be supplied in various manners, for instance in an alkaline solution, or in powder or granular form, or as a solid element such as a plate or rod or plurality of such elements. Metal supplied in solution will precipitate (and may be filtered or fall to a bottom of the reactor vessel) whereas metal carbonates formed on a solid element surface may be removed mechanically (e.g. by a scraping action), with vibration (e.g. using acoustic vibrations), or hydrodynamically (fluid forces) and collected at a bottom of the mineralization reactor vessel or by a filter mechanism, and removed with some of the anolyte liquid which may be replenished for instance in the recycling tank 32.

The recycled anolyte may be further processed in an anolyte recycling tank to keep the anolyte composition and pH with a specified range. The recycling tank may in particular comprise a water inlet 36 and an alkaline solution inlet 34, in particular for sodium hydroxide input, to replenish the analyte with water and sodium ions, before being recycled back through the anode via the anolyte inlet 49 thereof. Other metal hydroxides may be injected, as long as the corresponding metal carbonate thereof has a large solubility sufficient to prevent its precipitation in the AEM membrane. The sodium (or other metal ion of the selected hydroxide) ensures that the metal ions used for reaction with the carbonates and bicarbonate ions does not flow into anode and cause deterioration of the electrodes.

The cathode products output from the outlet 10o of the cathode 8 are fed to the methanation reactor 37 of the methanation system, and the carbon dioxide separated out of the anode products by the gas separator 40 is also fed to the methanation reactor. Additional hydrogen, for instance from a hydrogen source 6 comprising a water electrolyser 42 may further be connected to the inlet of the methanation system 4 in order to supply additional hydrogen for the production of synthetic natural gases. The amount of hydrogen is preferably adjusted to ensure a stochiometric balance in which all or most of the carbon dioxide supplied from the anode products is converted into fuel and oxygen.

The mineralization system 3 coupled to the anolyte circuit 18 of the anode 12 allows to use an anion exchange membrane for the co-electrolysis of CO₂ and H₂O in a particularly efficient manner, benefiting from the low temperature functioning of the AEM electrolysis while avoiding the detrimental effects of the carbonate and bicarbonate ions that pass through the membrane, and provide a stable capture and storage of CO₂ with the production of metal carbonates. The co-electrolysis of CO₂ and H₂O including feeding products into a methanation reactor further allows an overall globally efficient conversion of carbon dioxide and production of useful fuels therefrom (namely synthetic natural gas) in an energy efficient and economical process.

A description of the electrode reactions and products, as well as the mineralization reactions and products are provide in more detail below.

The ratio of CO₂/H₂O input into the cathode for a 100% water saturated CO₂ gas can vary in a range of 30 to 1 depending on the operating temperature, which may lie in a range from 25° C. to 140° C. The amount of water may however exceed 100% relative humidity, or be less than 100% relative humidity.

In the electrolysis cell there are a set of reactions in the CO₂/H₂O co-electrolysis that happen on both cathode and anode sides:

### Cathode Main Reactions:

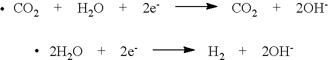

### Cathode Side Reactions:

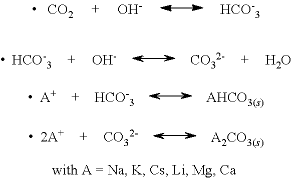

In a CO₂ rich environment carbonate (CO₃²⁻) and bicarbonate (HCO₃⁻) ions are formed at the cathode and can cross the membrane. Conversely, alkali metals ions can pass from anode to cathode and react with the carbonate and bicarbonate ions precipitating salts which could damage the membrane and decrease performance.

On the anode side, there is Oxygen evolution, some CO₂ regeneration, together with the mineralization reactions:

### Anode Main Reactions:

$$2 {OH}^{-} \to {H}_{2} O + \frac{1}{2} {O}_{2} + 2 {e}^{-}$$

### Anode Side Reactions:

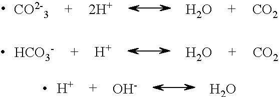

CO₂ is released at the anode due to unreacted carbonate and bicarbonate crossover.

### Overall Reactions:

$${CO}_{2} \to CO + \frac{1}{2} {O}_{2}$$

$${H}_{2} O \to {H}_{2} + \frac{1}{2} {O}_{2}$$

### Mineralization

The mineralization of the carbonate and bicarbonate ions generated by the two reactions below that occur on the anode side

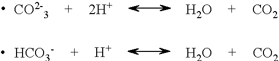

has a two-fold advantage: on the one hand the re-generation of CO₂ (the initial feedstock) is avoided and on the other hand useful minerals are co-produced.

Indeed, for mineralization, different metals can be used. Depending on its ion charge, the stoichiometry is different:

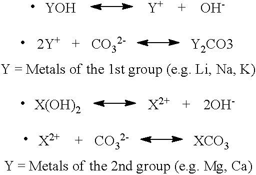

Other reactions are possible with transition metals, such as Mn, Fe, Co, Ni, Cu, Zn.

The inventors have simulated operating conditions at 60° C. and 1 bar, that lead to a complete conversion of Mg(OH)₂ into MgCO₃ (X═Mg). The reaction is exothermic, and heat needs to be evacuated. A sensitivity analysis as a function of temperature is provided in FIG. 3.

Mineralisation can be obtained with various metal ion sources, for instance:
1. Directly by using a solid metal rod, sheet, mesh, felt, fibre, foams, or other solid member form from a metal of the 1st or 2nd group of the periodic table. In such cases, the mineralization reaction occurs on the surface of the solid form, and the reaction follows:

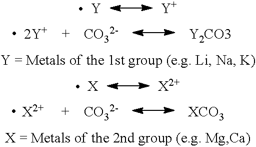

Similar reactions are possible with transition metals, such as Mn, Fe, Co, Ni, Cu, Zn;
2. Via an Oxide alternative, such as MgO, or CaO. In that case the reaction of the anioning species and the regeneration of the CO₂ can occur, as long as there is available Oxide for the reaction:

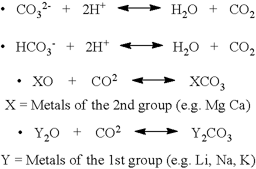

Similar reactions are possible with transition metals, such as Mn, Fe, Co, Ni, Cu, Zn.

The preferred metals for mineralization are selected from the second group or the transition metals, most preferred from the second group in particular Mg or Ca in view of the stability and practical usefulness of the resulting carbonates MgCO₃ and CaCO₃ in various industry applications such as in the building industry. Both MgCO₃ and CaCO₃ have applications in construction (agglomerates and aggregates of different type), road construction material (to be included in asphalt), land reclamation, fillers for different applications, ranging from biological applications to construction material. These materials are used due to their extreme thermodynamic stability, and relative abundance in the earth crust.

### Experimental Ranges and Conditions by Way of Example

### AEM Conditions—Examples

Voltage Range: 1.5 to 4V per electrolysis cell having a membrane electrode assembly (MEA) which could be stacked to form an electrolyser stack.
MEA area range: 0.1 to 4 m²
Current Density: 0-5000 mA cm⁻²
Membrane Thickness range: 2 to 200 μm.
Membrane Functionality and Materials: Commercial membranes such as X37-50 RT from Dioxide materials containing polystyrene methyl methylimidazolium chloride (PSMIM) or polystyrene tetramethyl methylimidazolium chloride (PSTMIM) and PiperION Membranes made up of functionalized poly (aryl piperidinium) polymer would be the preferred membrane materials, sandwiched between catalyst layers, incorporating zero gap electrolysis technology, as illustrated schematically in FIG. 1
Temperature: 0° C. to 140° C.
Faradaic efficiencies between 30-99.99%
Metals: Hydroxides, Carbonate and Bicarbonates of alkali/alkaline earth metals, transition metals and noble metals.
Electrolyte concentration: 0 to 60 wt %
Electrode substrates: foams, felts, fibres, meshes, sheets or plates made up of alkali group metals, transition metals, noble metals.
CO₂ flowrates: 0.05 to 160 L/min (or up to 4 ml/min cm⁻²) with relative humidification (RH) ranges from 0 to 100% with possibility supersaturated conditions (RH>100%)

Current Density (J)-Voltage (V) scans as illustrated in FIG. 4a to 4c is a characterization technique used to map the performance of MEA for listed reactions.

Total J: The total applied current density on the MEA resulting in a voltage response, or vice versa, resulting in the formation of syngas (CO+H₂) with residual (unconverted) CO₂.

JCO: The partial current density resulting in the formation of carbon monoxide species as a function of CO faradaic efficiency, as shown in equation (1).

$${FE}_{CO} = \frac{J CO}{TotalJ} \qquad ( 1 )$$

Also defined as:

$$J CO = \frac{nco \cdot F \cdot xco \cdot Fm}{A}$$

where nco are the electrons needed for CO₂ to CO electro-reduction, F is the Faraday constant (96,487 C/mol), xco is the mole fraction of CO in the product gas, Fm (mol/s) is the molar flow rate of the product gas and A is the cell's area.
JH2: The partial current density resulting in the formation of hydrogen species as a function of H₂ faradaic efficiencies, as shown in equation (2).

$${FE}_{H 2} = \frac{J H 2}{TotalJ}$$

Also defined as:

$$J H 2 = \frac{nh2 \cdot F \cdot xh2 \cdot Fm}{A}$$

where nh2 are the electrons needed for H₂O to H₂ electro-reduction, F is the Faraday constant (96,487 C/mol), xh2 is the mole fraction of H₂ in the product gas, Fm (mol/s) is the molar flow rate of the product gas and A is the cell's area.

The ratio's of partial current density is defined as shown in equation (3)

$$R = \frac{J CO}{J H 2}$$

Referring to FIGS. 5a to 5c, examples of different gas and energy inputs, and resulting outputs obtained from simulations, illustrate different use cases and configurations of the SNG production system, it being understood that preferred configurations may depend on the origin and availability of resources and the desired output products.

### LIST OF REFERENCED FEATURES

### Synthetic Natural Gas (SNG) Production System 100

CO₂ and H₂O Co-Electrolyser System 1
Electrolyser 2
   AEM Electrolyser cell 2C
      Cathode 8
         Inlet 10i
         Outlet 100
      Anode 12
         Anolyte Inlet 14i
         Anolyte Outlet 14o
         Anode products outlet 15
      AEM membrane 16
   Anolyte circuit 18
      Pump P
      Heat Exchanger HEX
   CO₂ inlet 20c
   H₂O inlet 20h
   CO₂ humidifier 22
Carbon dioxide (CO₂) source 24
Water/Steam (H₂O) source 26
Mineralization system 3
   Mineralization unit 27
   Mineralization metal inlet 28
   Carbonate outlet 30
Anolyte recycling tank 32
   Alkaline solution (e.g. sodium Hydroxide (NaOH)) inlet 34
   Water inlet 36
Anode products separation system 5
   Gas separator unit 40
      e.g. Pressure swing absorption (PSA) unit
      Anode products inlet 41i
      Oxygen Outlet 41o
Carbon dioxide (CO₂) source 24
Water/Steam (H₂O) source 26
   Methanation system 4
Methanation reactor 37
Gas fuel outlet 38
Valve V
CO₂ outlet 41c
   Hydrogen (H₂) source 6
Water electrolyser 42

1. A water and carbon dioxide co-electrolyser system comprising
an anion exchange membrane (AEM) electrolyser having at least one AEM electrolyser cell comprising a cathode, an anode and an AEM membrane separating the cathode from the anode,
an anolyte circuit in which an anolyte flows connected to the anode via an anolyte inlet and anolyte outlet of the anode,
the CO₂ and H₂O co-electrolyser system further comprising
a mineralization system including a mineralization unit connected to the anolyte circuit and containing a mineralization metal configured to react with the carbonate and bicarbonate ions circulating in the anolyte circuit to form a metal carbonate.

2. The system according to claim 1 wherein the anolyte circuit forms a closed circuit for recycling the anolyte solution.

3. The system according to the claim 2 wherein the anolyte circuit comprises an anolyte recycling tank having a water inlet and an alkaline solution inlet for replenishing the anolyte.

4. The system according to the claim 3 wherein the mineralization system comprises a metal carbonate deposit outlet at a bottom of a vessel of the mineralization unit for removal of metal carbonate deposits from the mineralization unit.

5. The system according to claim 1 wherein the mineralization system comprises a mineralization metal inlet configured for injection of a solution containing metal ions into the anolyte circuit for reaction with the carbonate and bicarbonate ions to produce a metal carbonate that is precipitated out of the anolyte solution.

6. The system according to claim 1 wherein the mineralization metal is provided in the form of a solid element such as a rod, bar, plate, or mesh.

7. The system according to claim 1 wherein the mineralization metal is selected from a group of metals consisting of Li, Na, K, Mg, Ca, Mn, Fe, Co, Ni, Cu, Zn.

8. The system according to claim 7 in which the mineralization metal is selected from a group of metals consisting of Mg, Ca, Mn, Fe, Co, Ni, Cu, Zn.

9. The system according to claim 8 in which the mineralization metal is selected from a group of metals consisting of Mg, Ca.

10. The system according to claim 1 wherein the AEM membrane comprises one or more layers of polystyrene methyl methylimidazolium chloride (PSMIM) or polystyrene tetramethyl methylimidazolium chloride (PSTMIM) or functionalized poly (aryl piperidinium) polymer, sandwiched between one or more catalyst layers.

11. The system according to claim 1 wherein the AEM electrolyser anode comprises an anode products outlet connected to a gas separator, for instance comprising a pressure swing absorption (PSA) unit for separating carbon dioxide and oxygen output from the anode.

12. The system according to claim 1 wherein the cathode is connected to a carbon dioxide source and a water source that are connected to a humidifier for injecting humidified carbon dioxide into the cathode side of the AEM electrolyser.

13. A synthetic natural gas (SNG) production system comprising a carbon dioxide and water co-electrolyser system according to claim 1 and further comprising a methanation system including a methanation reactor connected to the outlet of the cathode and an outlet of the gas separator outputting carbon dioxide.

14. The system according to claim 13 further comprising a hydrogen source including a water electrolyser connected to a renewable energy electrical source.
